(19) United States
(12) Patent Application Publication
(10) Pub. No.: US 2008/0081940 A1
(43) Pub. Date: Apr. 3, 2008

(54) ENERGY PATCH FOR CONTROLLING ENERGY AND BLOOD OF HUMAN BODY AND THERAPY METHOD USING THE SAME

(76) Inventor: In-Taek BYUN, Seoul (KR)

Correspondence Address: IPLA P.A., 3580 WILSHIRE BLVD., 17TH FLOOR, LOS ANGELES, CA, 90010, US

(21) Appl. No.: 11/555,159

(22) Filed: Oct. 31, 2006

(30) Foreign Application Priority Data
Sep. 12, 2006 (KR) 10-2006-0087811

(51) Int. Cl. A61B 17/52; A61N 2/00

(52) U.S. Cl. 600/9

(57) ABSTRACT

An energy patch for controlling energy and blood of a human body and a therapy method using the same are disclosed, which has a tonification and sadation so that it is adapted to a meridian and acupuncture point of a human body and is easily used. The energy patch for controlling energy and blood of a human body comprises a rubber alloy magnet which has a permanent magnet characteristic and is formed in a plane shape with one sharp corner being formed in a lower direction of the same, and at least one sharp corner being formed in an upper direction of the same; a color coating member which is color-printed on at least one surface of the surfaces of the rubber alloy magnet; and an adhering member for attaching at an acupuncture point of a human body.

### CROSS REFERENCE

Applicant claims foreign priority under Paris Convention and 35 U.S.C. §119 to the Korean Patent Application No. 10-2006-0087811, filed Sep. 12, 2006 with the Korean Intellectual Property Office.

### BACKGROUND OF THE INVENTION

1. Field of the Invention

The present invention relates to an energy patch for controlling energy and blood of a human body and a therapy method using the same, and in particular to an energy patch which is attached to a certain portion of a human body in consideration with a compass direction energy and a chromatic energy of a human body for thereby concurrently achieving a magnetic therapy effect using a magnet and an acupuncture effect.

2. Description of the Background Art

In the descriptions of the present invention, an energy patch is referred to an attachable patch which is made of a thin flat rubber alloy magnet and implements a smooth circulation of energy by attaching on a skin of a human body using a certain attaching member such as a plaster.

According to a traditional oriental medical field, an acupuncture therapy is considered as an important therapy method. Here, the acupuncture method is implemented based on a meridian theory. In the above method, a certain therapy is obtained by treating a needle at a meridian corresponding to a path through which energy circulates in a human body and at an acupuncture point having special functions.

The human body comprises 12 meridians, with each meridian representing an internal organ function and being capable of balancing and controlling weakness and strength, cold and heat, so that a good mental health and physical health are obtained. In order to obtain a good implementation of meridians, five acupuncture points of each meridian is needed to be well treated. According to a traditional oriental medical field, energy is directly related with water. The energy of Chinese character is made based on a phenomenon that vapor rises. When the vapor rises, it becomes rain, and the rain makes a river. The river provides a human body with water. According to an ancient saint, a good circulation of water allows a human to live well. 70% of a human body is formed of water. “A good energy circulation” equals to “a good water circulation”. According to a traditional oriental acupuncture therapy, the circulations of energy at 12 meridians are controlled using the above energy circulation theory.

According to the distribution of meridians, the five acupuncture points are located at the ends of hands and legs, namely, are located below knee joint and have five day-based attributes of Thursday, Thursday, Saturday, Friday and Wednesday. According to a traditional oriental medical theory, the five days react with compatibility and incompatibility for thereby allowing a life to live forever. An acupuncture method capable of controlling weakness and strength of internal organs based on compatibility and incompatibility of five acupuncture points has been brought, so that it is widely researched by clinical doctors as including an oriental medical doctor.

The magnetic needle therapy attracts a big attention based on its functions of inducing a magnetic field at a meridian and acupuncture point of a human body using an electromagnetic induction phenomenon instead a conventional needle, for thereby easily detecting a meridian and acupuncture point, so that it is possible to cure a certain disease and enhance a health of people.

As a prior art of the present invention, there is provided a Korean utility model publication number 20-0293144 “Magnetic needles for stimulating meridians and acupuncture points”. FIG. 1 is a perspective view illustrating a magnetic needle of the registered Korean utility model publication number 20-0293144. As shown therein, the magnetic needle 1 is formed in a cylindrical disk shape like an old Korean traditional coin or a donut. A cylindrical through hole 2 is formed in the interior of the same, so that magnetic force lines pass through the through hole 2 for thereby condensing magnetic flux.

However, the magnetic needle of the conventional art is attached to a skin of a human body by adhering an adhering band to a cylindrical disk. Since the magnetic needle is simply attached to a certain portion of a human body and is used, a compass direction energy or a chromatic energy as needing an energy and blood control of a human body while selectively treating a needle like a traditional oriental medical field is not adapted to the above conventional art, so that the effects of needle decrease.

As another prior art of the present invention, as shown in FIG. 2, there is provided a Korean utility model publication number 1998-056261 “Isotropic magnetic patch”.

As shown in FIG. 2, a spring needle 3 is inserted into a through hole 4 of a circular magnet 5, and a lower end of the circular magnet 5 is covered by a specially plated cap 6, so that the spring needle 3 inserted into the through hole 4 is provided with magnetic force. The generated magnetic force activates an operation of Hemoglobin contained in blood as circulating in a human body. Reference numeral 7 represents an adhering band.

Here, the magnetic needle has a magnetic force of 600 Gauss±10% at its N-pole and S-pole. In particular, since the magnetic needle is formed of an isotropic structure, multiple needles may be concurrently used for treatment.

A circular magnet made of an adhering band and a ferrite magnet is treated at a blood vessel using a spring needle in a through hole formed at the front end. A specially plated cap is covered at a lower end of the circular magnet for thereby treating at a meridian point based on its isotropic structure so that magnetic force lines are concentrated at a certain portion. The magnetic force lines affect a biological current of a human body and Hemoglobin of blood for thereby activating an energetic behavior.

However, in the case of a conventional magnetic needle, the magnetic needle is used in such a manner that the spring needle 3 is inserted into the through hole 4 of the circular magnet 5 and is attached to a skin of a human body using a band. Since the magnetic needle is simply attached to a certain portion of a human body and is used, it does not have a tonification and sadation function, which is known to provide an energy to a weak energy portion by selectively treating a needle like an oriental acupuncture therapy and which releases a strong energy portion, so that an energy control ability decreases.

As another problem of the conventional art, since a magnetic needle is too thick or heavy, it is not well attached to a skin of a human body. When it contacts with a skin of a human body, a certain skin trouble may occur.

### SUMMARY OF THE INVENTION

Accordingly, it is an object of the present invention to provide an energy patch for controlling energy and blood of a human body and a therapy method using the same which overcome the problems encountered in the conventional art.

It is another object of the present invention to provide an energy patch for controlling energy and blood of a human which has a tonification and sadation so that it is adapted to a meridian and acupuncture point of a human body and is easily used.

It is further another object of the present invention to provide an energy patch for controlling energy and blood of a human body in which at least sharp corner is provided at upper and lower corner portions of a rubber alloy magnet, so that a pyramid effect is concentrated at a corner portion of a needle, and it is possible to effectively control an acupuncture point by treating at a skin of a human body in consideration with a compass direction and chromatic characteristic of an energy patch depending on a user.

It is still further another object of the present invention to provide a therapy method for curing common cold, fatigue, atopic dermatitis, cough, bronchial asthma and constipation using an energy patch.

To achieve the above objects, in an energy patch for controlling energy and blood based on a tonification and sadation method with respect to energy and blood at meridians of a human body, there is provided an energy patch for controlling energy and blood of a human body which comprises a rubber alloy magnet which has a permanent magnet characteristic and is formed in a plane shape with one sharp corner being formed in a lower direction of the same, and at least one sharp corner being formed in an upper direction of the same; a color coating member which is color-printed on at least one surface of the surfaces of the rubber alloy magnet; and an adhering member for attaching at an acupuncture point of a human body.

In the best mode for carrying out the present invention, the rubber alloy magnet forms one sharp corner in a lower direction of the same, and a sharp center corner is formed in an upper direction of the same, and two sharp corners are spaced-apart and formed at both sides of the center corner.

The color of the color coating member is selected among white, black, blue and red colors based on a corresponding zodiac sign among twelve zodiac signs of a human body.

Preferably, the persons of the tiger, horse and dog zodiac signs have an activation color of black. The persons of the pig, rabbit and sheep zodiac signs have an activation color of white. The persons of the monkey, rat and dragon zodiac signs have an activation color of red. The persons of the snake, chicken and cow zodiac signs have an activation color of blue.

The method for curing a common cold, fatigue, constipation, atopic dermatitis, and bronchial asthma comprises a step for determining a compass direction energy based on twelve zodiac signs with respect to a patient's lunar calendar age, a step for determining an acupuncture point for curing a corresponding disease and a position of the acupuncture point, a step for selecting a color of the energy patch and a step for attaching an energy patch at the selected acupuncture point.

### BRIEF DESCRIPTION OF THE DRAWINGS

The present invention will become better understood with reference to the accompanying drawings which are given only by way of illustration and thus are not limitative of the present invention, wherein;

FIG. 1 is a perspective view illustrating a conventional energy patch;

FIG. 2 is a disassembled perspective view illustrating a conventional isotropic magnetic needle patch;

FIG. 3 is a front view illustrating an energy patch according to a first embodiment of the present invention;

FIG. 4 is a cross sectional view of FIG. 3;

FIG. 5 is a front view illustrating an energy patch according to a second embodiment of the present invention;

FIG. 6 is a front view illustrating an energy patch according to a third embodiment of the present invention;

FIG. 7 is a front view illustrating an energy patch according to a fourth embodiment of the present invention;

FIG. 8 is a front view illustrating an energy patch according to a fifth embodiment of the present invention;

FIG. 9 is a front view illustrating an energy patch according to a sixth embodiment of the present invention;

FIG. 10 is an exemplary view of a method of use according to the present invention;

FIG. 11 is a view illustrating a product of an energy patch according to the present invention;

FIGS. 12A through 12C are views for describing a method for curing common cold using an energy patch according to the present invention;

FIGS. 13A through 13C are views for describing a method for curing fatigue using an energy patch according to the present invention;

FIGS. 14A through 14D are views for describing a method for curing atopic dermatitis using an energy patch according to the present invention;

FIGS. 15A through 15C are views for describing a method for curing bronchial asthma using an energy patch according to the present invention; and

FIGS. 16A through 16E are views for describing a method for curing constipation using an energy patch according to the present invention.

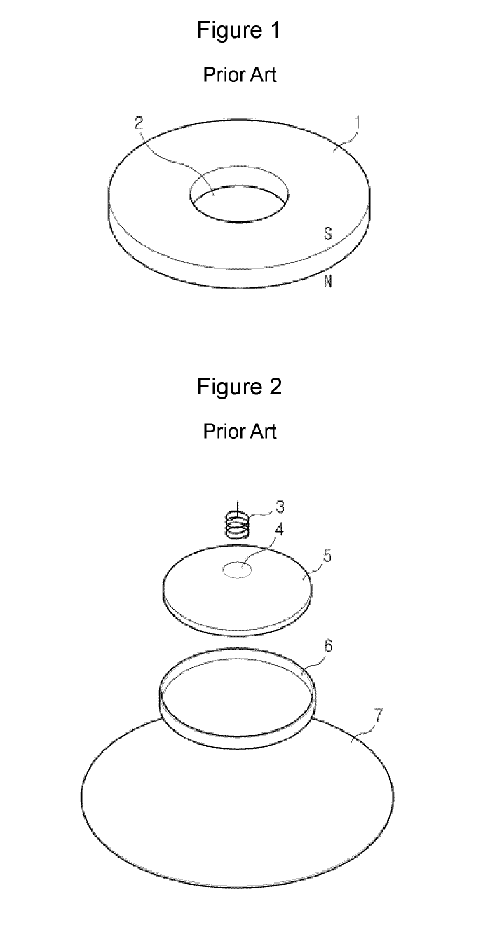

### DETAILED DESCRIPTION OF THE PREFERRED EMBODIMENTS

The preferred embodiments of the present invention will be described with reference to the accompanying drawings.

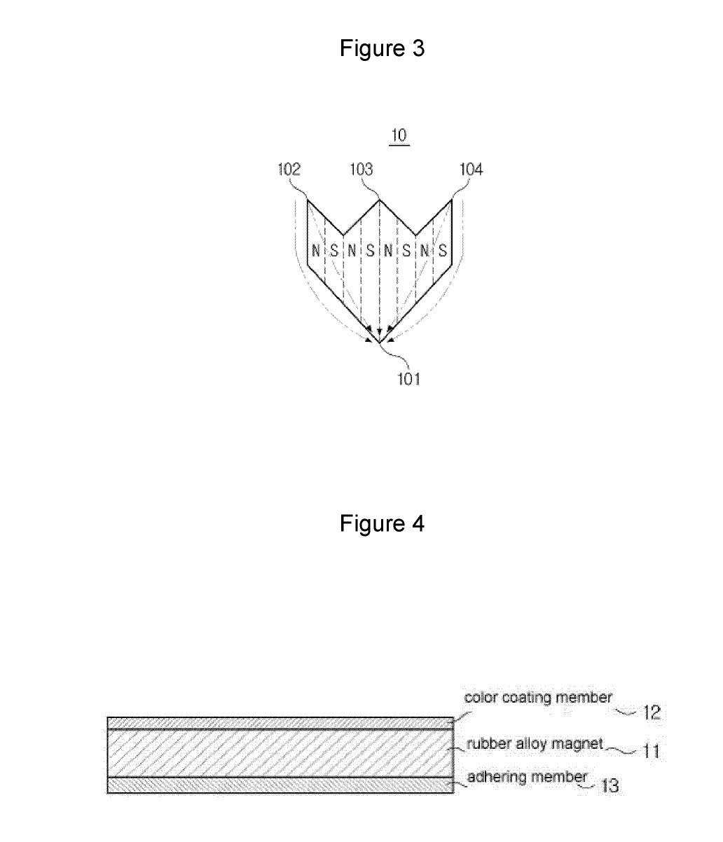

FIG. 3 is a front view illustrating an energy patch according to a first embodiment of the present invention. FIG. 4 is a cross sectional view of FIG. 3.

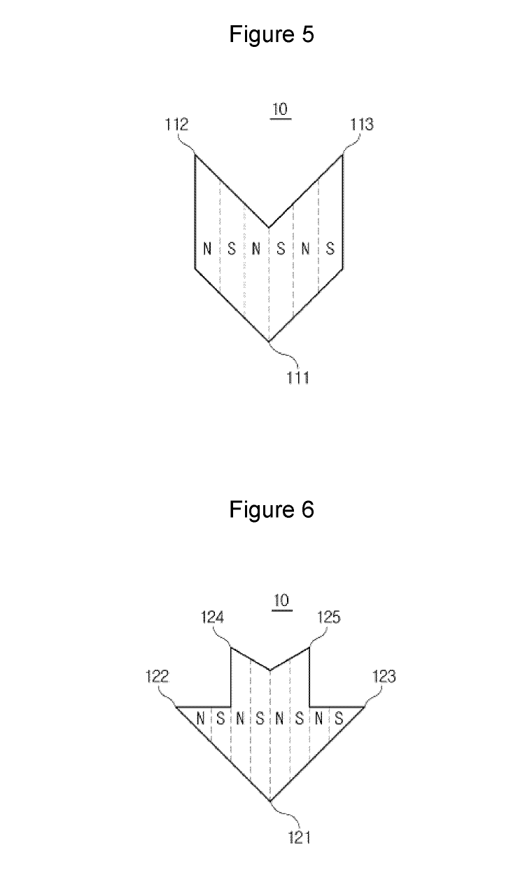

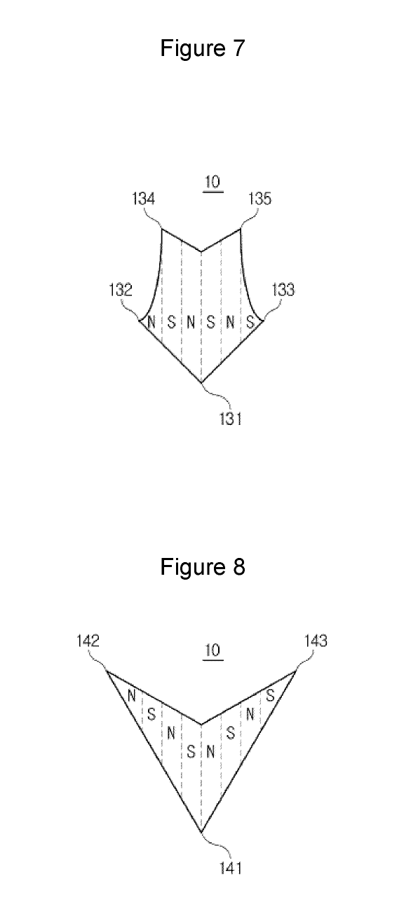

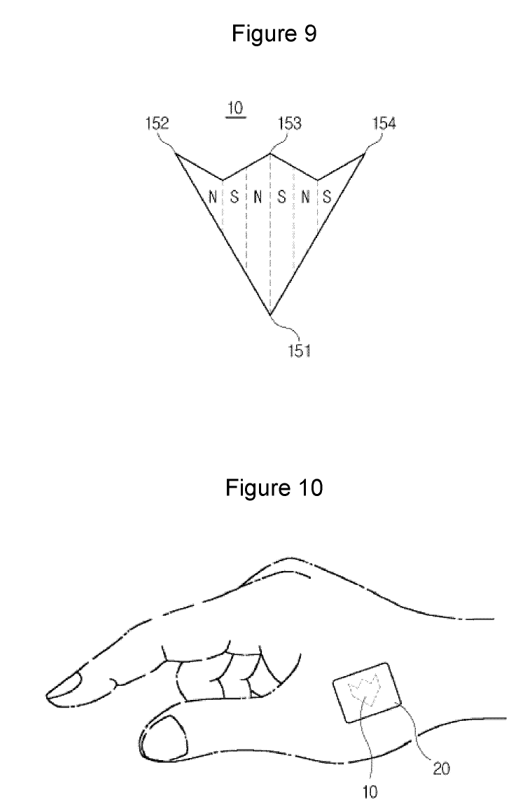

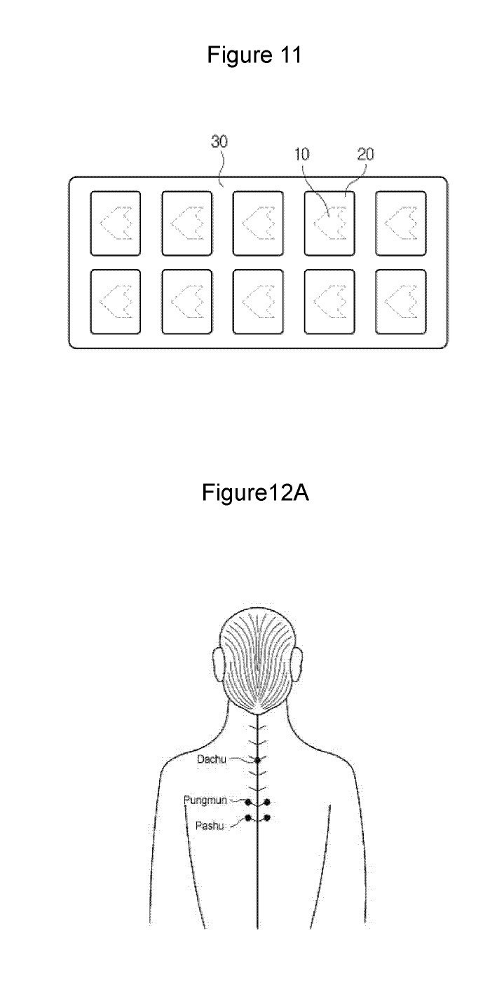

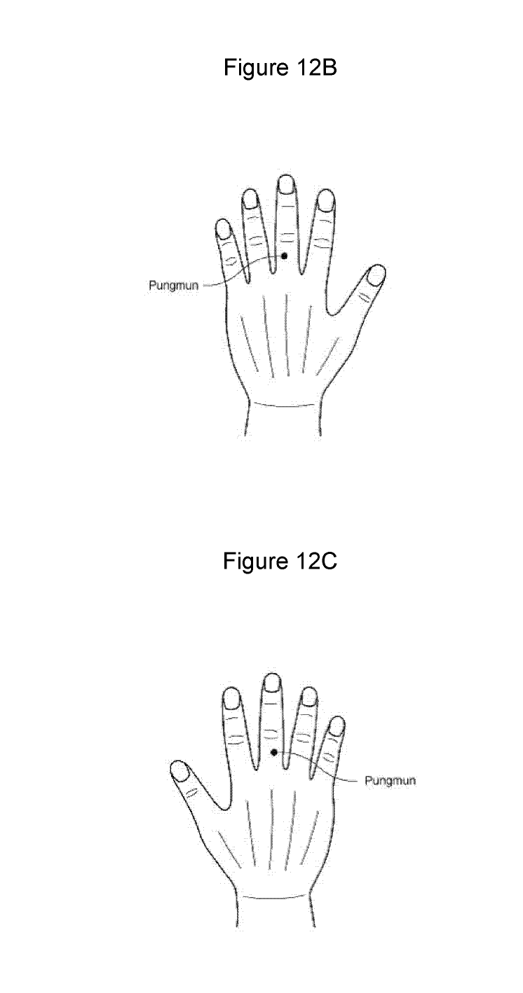

FIG. 5 is a front view illustrating an energy patch according to a second embodiment of the present invention. FIG. 6 is a front view illustrating an energy patch according to a third embodiment of the present invention. FIG. 7 is a front view illustrating an energy patch according to a fourth embodiment of the present invention. FIG. 8 is a front view illustrating an energy patch according to a fifth embodiment of the present invention. FIG. 9 is a front view illustrating an energy patch according to a sixth embodiment of the present invention. FIG. 10 is an exemplary view of a method of use according to the present invention. FIG. 11 is a view illustrating a product of an energy patch according to the present invention. FIGS. 12A through 12C are views for describing a method for curing common cold using an energy patch according to the present invention.

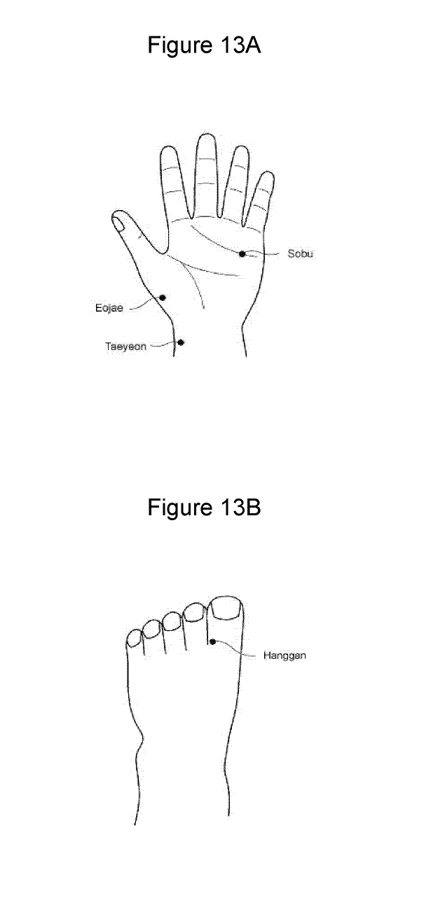

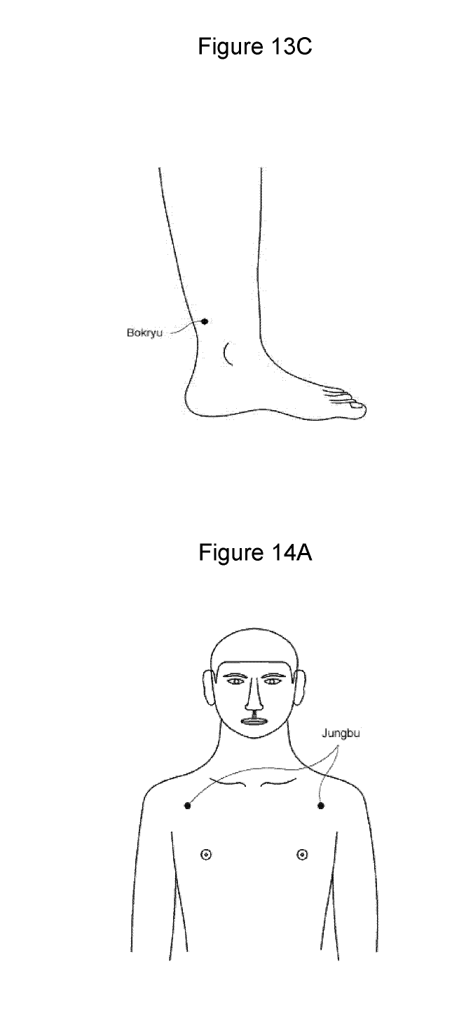

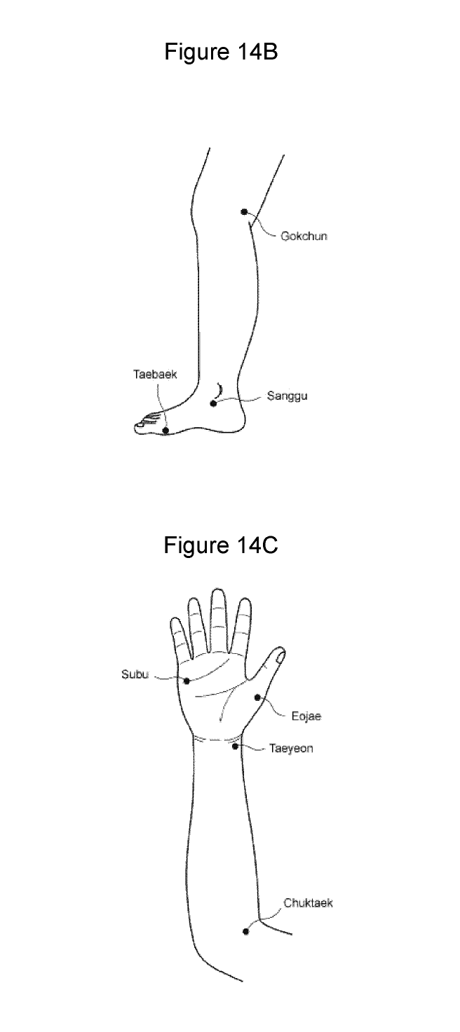

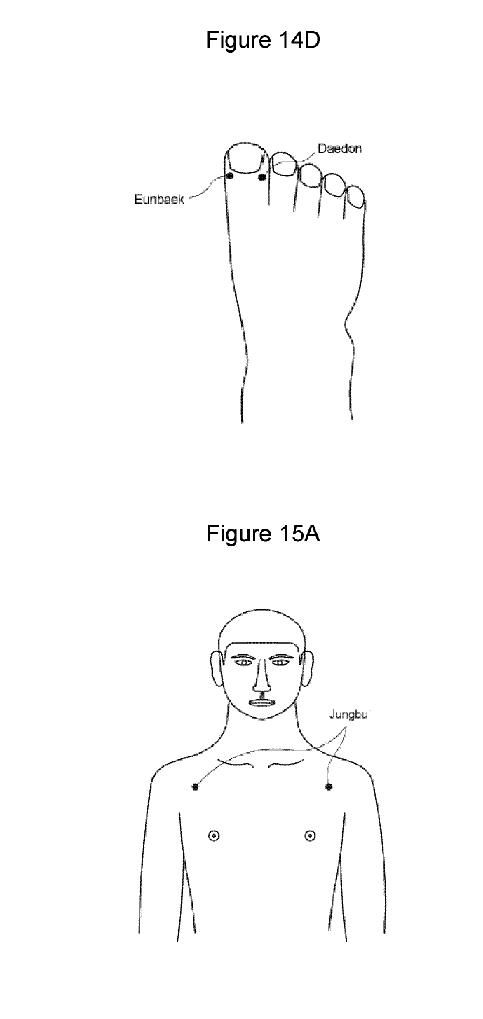

FIGS. 13A through 13C are views for describing a method for curing fatigue using an energy patch according to the present invention. FIGS. 14A through 14D are views for describing a method for curing atopic dermatitis using an energy patch according to the present invention.

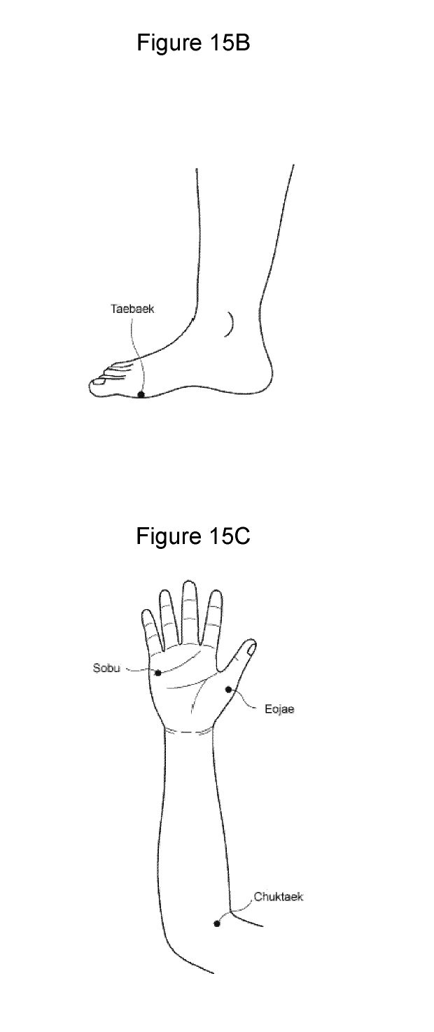

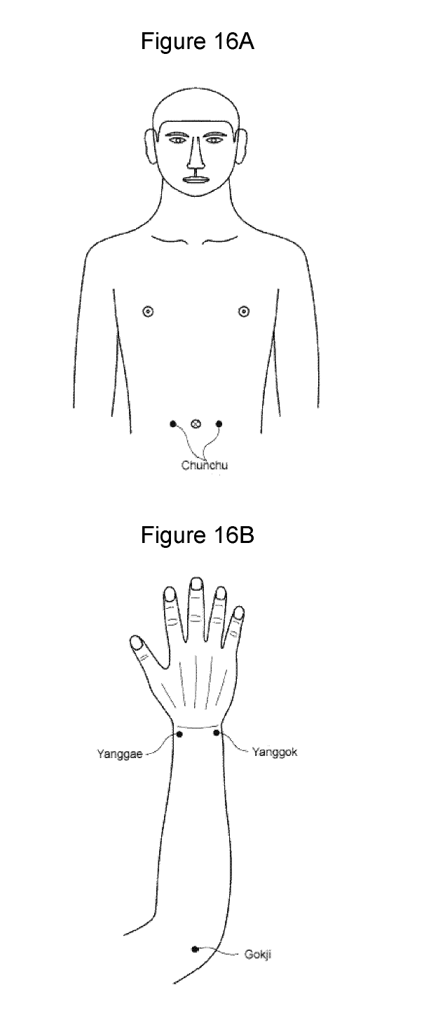

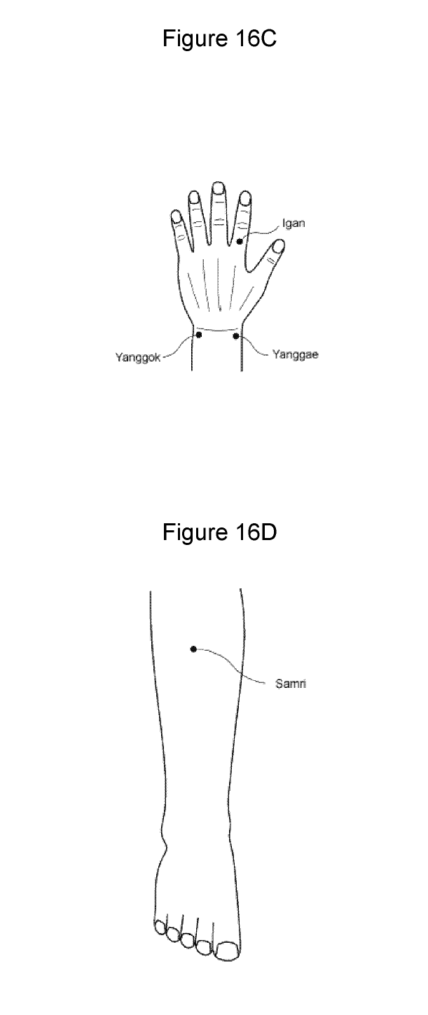

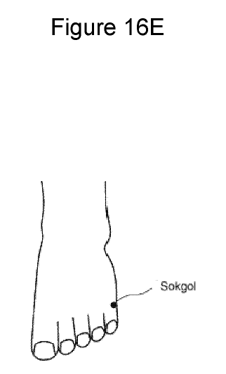

FIGS. 15A through 15C are views for describing a method for curing bronchial asthma using an energy patch according to the present invention. FIGS. 16A through 16E are views for describing a method for curing constipation using an energy patch according to the present invention.

FIG. 3 is a front view illustrating an energy patch according to a first embodiment of the present invention, and FIG. 4 is a cross sectional view of FIG. 3.

In an energy patch for controlling energy and blood based on a tonification and sadation method with respect to energy and blood at meridians of a human body, an energy patch for controlling energy and blood of a human body comprises a rubber alloy magnet 11 which has a permanent magnet characteristic and is formed in a plane shape with one sharp corner being formed in a lower direction of the same, and at least one sharp corner being formed in an upper direction of the same, a color coating member 12 which is color-printed on at least one surface of the surfaces of the rubber alloy magnet 11, and an adhering member 13 for attaching at an acupuncture point of a human body.

The rubber alloy magnet 11 forms one sharp corner 101 in a lower direction of the same, and a sharp center corner 103 is formed in an upper direction of the same, and two sharp corners 102 and 104 are spaced-apart and formed at both sides of the center corner 103.

A color of said color coating member 12 is printed by selecting among white, black, blue and red colors based on a corresponding zodiac sign among twelve zodiac signs of a human body.

When selecting the color of the color coating member 12, the tiger, horse and dog zodiac signs are provided with a black color, respectively, and the pig, rabbit and sheep zodiac signs are provided with a white color, respectively, and the monkey, rat and dragon zodiac signs are provided with a red color, respectively, and the snake, chicken and cow zodiac signs are provided with a blue color, respectively.

The operation and effect of the present invention will be described.

As shown in FIGS. 3 through 9, the energy patch according to the present invention comprises a tonification and sadation effect based on an electric field formed between the N-pole and the S-pole, a pyramid effect in which an energy is concentrated at a sharp portion of the energy patch, and a compass direction energy and chromatic energy which are considered when an energy patch is treated at a skin of a human body.

The energy patch according to the present invention is characterized in that magnetic force lines are concentrated at a lower corner portion in an upper sharp corner direction. In the present invention, it is possible to achieve a tonification and sadation effect by attaching the above energy patch at a skin of a human body.

The technical term “tonification and sadation” of the present invention represents that a shortage portion is supplemented, and a too much portion is reduced. The principle of the tonification and sadation is implemented based on an operation that a magnetic force line is defined toward a lower corner portion. The effects of tonification and sadation can be obtained by guiding the energy of meridian, namely, energy based on the flow of magnetic force. When the patch is attached at an acupuncture point (five acupuncture points) with an upper direction corner being upward and a lower direction corner being downward, the energy of the acupuncture point is supplemented based on the flow of magnetic force for thereby achieving a tonification effect.

On the contrary, when the patch is attached at an acupuncture point with a lower direction corner being upward and an upper direction corner being downward, the energy of the acupuncture point is reduced based on the flow of magnetic force for thereby achieving a sadation effect. The present invention is capable of overcoming the limit of conventional acupuncture treatment by attaching a patch according to the present invention at an acupuncture point and is capable of maximizing a duration time of acupuncture effect.

According to the energy patch according to the present invention, since energy is concentrated at a sharp corner of a lower direction of the same, a pyramid effect can be obtained. According to the pyramid effect, when a person thinks or sleeps within the pyramid, biological energy is activated, and head feels refresh, and concentration is enhanced.

A mysterious point of the pyramid as built in 2700BC lies in a direction of a huge structure. Each corner ridgeline accurately indicates East, West, South and North, respectively. The structure of a pyramid allows energy to be concentrated and to radiate for thereby generating a lot of α-wave among brain waves. Many scientists verify that a higher amplitude of α-wave is generated as a result of a brain wave measurement experiment which is performed within a pyramid with a brain wave detection apparatus being attached to a header of a meditator.

FIG. 5 is a front view illustrating an energy patch according to a second embodiment of the present invention. FIG. 6 is a front view illustrating an energy patch according to a third embodiment of the present invention. FIG. 7 is a front view illustrating an energy patch according to a fourth embodiment of the present invention. FIG. 8 is a front view illustrating an energy patch according to a fifth embodiment of the present invention. FIG. 9 is a front view illustrating an energy patch according to a sixth embodiment of the present invention.

The energy patches of FIGS. 3 through 9 have the common features, which lie in a sharp corner shape like a pyramid. The common features are as follows. Namely, as shown in FIG. 3, the materials of the energy patches are isotropic rubber alloy magnets, and the shapes of the same are magnet needles formed of thin flat rubber alloy magnets. The N-pole and S-pole are arranged as shown in the figures, so that magnetic force lines are generated and concentrated from an upper direction to a lower direction.

In particular, one sharp corner is formed in the lower direction of the same like an apex of a pyramid, and at least one sharp corner is formed in an upper direction or at a center portion of the same.

As shown in FIG. 5, the rubber alloy magnet 11 forms one sharp corner 111 in a lower direction of the same, and sharp corners 112 and 113 are symmetrically formed at both sides of an upper direction of the same.

As shown in FIG. 6, the rubber alloy magnet 11 forms one sharp corner 121 in a lower direction of the same, and two corners 122 and 123 are spaced-apart and formed at both sides of the center corner. Sharp corners 124 and 125 are symmetrically formed at both sides of an upper direction of the same.

The construction of FIG. 7 is similar with the construction of FIG. 6. The rubber alloy magnet 11 forms one sharp corner 131 in a lower direction of the same, and two corners 132 and 133 are spaced-apart and formed at both sides of a center corner. Sharp corners 134 and 135 are symmetrically formed at both sides of an upper direction of the same.

As shown in FIG. 8, the rubber alloy magnet 11 forms one sharp corner 141 in a lower direction of the same. Sharp corners 142 and 143 are symmetrically formed at both sides of an upper direction of the same.

As shown in FIG. 9, the rubber alloy magnet 11 forms one sharp corner 151 in a lower direction of the same. A sharp center corner 153 is formed in an upper direction of the same. Two sharp corners 152 and 154 are spaced-apart and formed at both sides of the center corner 153. The shape of the corner is similar with the shape of FIG. 3 except that an inclination portion linking each corner is different.

The energy patch according to the present invention may be formed of a common permanent magnet, cloth or paper instead a rubber alloy magnet. In a preferred embodiment of the present invention, the energy patch is designed to generate magnetic force of about 2000 through 4000 Gauss. In a more preferred embodiment of the present invention, a permanent magnet capable of generating a magnetic flux intensity of about 300 Gauss may be used. The thickness of a rubber alloy magnet is preferably within a range of 0.5 mm to 5.0 mm. In a most preferred embodiment of the present invention, the thickness of a rubber alloy magnet is 0.8 mm.

FIG. 10 is an exemplary view of a method of use according to the present invention. As shown therein, the energy patch 10 according to the present invention is attached to a skin of a human body using a fixing member 200. The fixing member 200 may be formed of a plaster, a medical tape or a patch. The key principle of the present invention lies in that when the energy patch 10 is attached to a skin of a human body, the attaching direction of the energy patch and the color of a color coating member 12 attached on a surface of the energy patch are determined based on 12 zodiac signs of a person.

The principles of the compass direction energy and the chromatic energy will be described.

1) Principle of Compass Direction Energy

The compass direction energy represents an energy which reacts when a person stands in a certain direction or reacts with a human body in the above direction. The chromatic energy represents an energy that a color reacts with a fatigue of a human body. A physical constitution recently attracts a big attention. It is judged that the physical constitution does largely affects an application of the compass direction energy and chromatic energy. So, the compass direction energy reacts identically with respect to any types of physical constitution.

The O-ring test is performed so as to check a non-harmful thing and a harmful thing in the expression of plus and minus with respect to a current body state of a testee.

Geographically, the brain of a testee checks a too much and a shortage of an energy and reacts in the expression of plus and minus by driving a constancy mechanism so as to maintain a balance between the too much and the shortage. The sensor(detection function) of the brain may be a certain obstacle with respect to the energy.

There are four compass direction energies and four chromatic energies. It is possible to expect an accuracy (reproduction) of the 0-ring test by properly combining the above eight energies. The above principle may be adapted for various applications such as an adult disease as well as a disease prevention and accurate diagnosis and therapy.

According to the five phases of Yin and Yang as used in the oriental medical field, the south direction represents tree, the west direction represents gold, the south direction represents fire, and the north direction represents water.

In the earth, there are cold latitudes and tropics. The above directional tendency has a very strong power which provides a physical constitution of a person with fire energy, tree energy, water energy and gold energy, respectively. The following table is a classification table of the compass direction energies according to the present invention, which is obtained based on a clinical result by the inventor.

TABLE

| 12 zodiac sign | Energy zodiac sign | Compass direction energy | Property of compass direction energy |
|---|---|---|---|
| Tiger, horse, dog | Fire zodiac sign | Fire compass direction energy | Straight only in North |
| Pig, rabbit, sheep | Tree zodiac sign | Tree compass direction energy | Straight only in West |
| Monkey, rat, dragon | Water zodiac sign | Water compass direction energy | Straight only in South |
| Snake, chicken, cow | Gold zodiac sign | Gold compass direction energy | Straight only in East |

In the above table, the 12 zodiac signs each represent a yearly zodiac sign in which a person is born. In addition, the 12 zodiac signs represent 12 earth zodiac signs which are below the sexagenary cycle in the oriental science while symbolically representing 12 animals.

According to the oriental science, it is known that the year, in which a person is born, represents one zodiac sign among 12 animals. Here, the 12 zodiac signs are rat, cow, tiger, rabbit, dragon, snake, horse, sheep, monkey, chicken, dog, and pig. For example, when a person is born in 1955, the person is given a sheep zodiac sign according to the 12 zodiac signs.

Here, the energy zodiac signs become one five-phase as formed of three earth zodiac signs of corresponding birth years. The compass direction energy does not disappear or does not change its property until a corresponding life is died. In the east direction, a tree energy band exists, and in the west direction, a gold energy band exists, and in the south direction, a fire energy band exists, and in the north direction, a water energy band exists. In each corresponding energy band, a certain energy having its inherent property exists in a certain direction.

The four compass direction energies have their inherent directions without any interferes even when there are a few obstacles and flow in a straight direction. For example, their inherent properties are maintained even when in a very small room having a certain space.

The rhythm of the compass direction energy has a relatively strong magnetic field for 365 days and then returns its ordinary state in the following year.

All things in the world have their inherent magnetic fields and magnetic waves. For example, in the case of the fire zodiac sign, a new born baby is provided with an intensive fire energy through head apex, brain, middle of eyebrows, thymus, central nerve, spinal cord, etc as the baby first breaths based on a brain breathing method, not based on a umbilical cord breathing method. The compass direction energy is penetrated into a newly born baby body like a certain pigment colors a piece of cloth or a sheet of paper.

Namely, one magnetic substance is born. At the same time, a huge size compass is formed in a human body magnet substance. When the hands of the compass indicate a direction of south→north, the movements of the hands are stopped.

In the case of the tree zodiac sign, when the hands of the compass of the tree zodiac sign indicate a direction of east→west, in other words, when it orients west, the movements of the hands are stopped.

The movements of the hands of the compass are respectively stopped when the water energy of north is received in such a manner that the hands indicate north→south in the case of the water zodiac sign, and the hands indicate west→east in the case of the gold zodiac sign, and the hands indicate south→north in the case of the fire zodiac sign.

When the magnetic direction(activation direction) of the hands of the compass is deviated, the hands move so as to search a certain magnetic direction. In worse case, an erroneous operation may occur in the operation of the brain, in particular in the detection operation of the sensor.

According to the principle of the O-ring test, the inherent functions of the O-ring test do not work properly due to over loads while the testee or tester is moving a lot, so that it is impossible to obtain a constant result of the operation.

2) Principle of Chromatic Energy

All substances have electromagnetic waves. A color has its own temperature and generates an inherent electromagnetic wave.

For example, the blue color generates a gold energy, the while color generates a tree energy, the red color generates a water energy, and the black color generates a fire energy.

The wave motion and wavelength of the color have astonishing powers. When a piece of paper or cloth of a small size of 5 mm is attached at a certain acupuncture point, a huge reaction occurs. Namely, an allergy or nasitis patient stops running at the nose, and coughing is stopped, and sleeplessness changes to a good sleep.

For example, when one side of a sheet of paper or cloth is red, and the other side of the same is black, a person of the fire zodiac sign may be provided with energy at underwear, sleep clothes, or socks when black color contacts with a skin of the person. When the colors of the outer clothes as not contacting with the skin do not affect the energy.

According to the analysis of the 12 zodiac signs, the persons of the tiger, horse and dog zodiac signs have an activation color of black. The persons of the pig, rabbit and sheep zodiac signs have an activation color of white. The persons of the monkey, rat and dragon zodiac signs have an activation color of red. The persons of the snake, chicken and cow zodiac signs have an activation color of blue. When the O-ring test is performed with an underwear of an activation color, socks of an activation color, and a posture of receiving an activation compass direction energy, the same results are obtained. The O-ring test is a method of quantifying and judging the energy balance of the testee at the current situation.

The brain of the testee well recognizes a too much and shortage of his own energy and informs the current state in the expression of plus and minus for indicating shortage(constancy) for recovery of energy balance. In other words, when the gold energy is less, the gold energy substance is expressed in the form of plus. The plus represents a minus state as having less gold energy. For example, when the left lung has 70 as compared to the normal value of 100, the value of 30 is supplemented (plus) for thereby balancing and obtaining the value of 100.

According to a result of the statistic obtained through a clinic test, the left lug of a person is weak, and the right lug is strong irrespective of human kinds and physical constitution. Since the left lug is weak, it means a minus state. When the energy of the right lug is 70 as compared to the normal value of 100, it is needed to add 30 (plus) for thereby balancing the weak and strong states to have 100. As a result of the O-ring test with respect to the weak and strong relationship, it is known that the grasping force of fingers increases (plus) so as to supplement (plus) the weak level of 30.

When the compass direction energy and chromatic energy are adapted in the same manner as the above description, namely, when a skin of a person is influenced by an activation color, and a underwear and stocks as contacting with a skin of a human body are worn, the strengths of the energy of our body may react accordingly.

The methods for curing diseases are cured using an energy patch for controlling energy and blood of a human body will be described according to the present invention. In the present invention, it is possible to cure various diseases using an energy patch according to the present invention. The applicant of the present invention, an oriental medical doctor, has obtained many good results of clinics based on the actual clinical backgrounds which are performed with respect to patients suffering from common cold, fatigue, atopic dermatitis, cough, bronchial asthma, constipation, etc.

The therapy methods according to the present invention comprises a step for determining a compass direction energy based on twelve zodiac signs with respect to a patient's lunar calendar age, a step for determining an acupuncture point for curing a corresponding disease and a position of the acupuncture point, a step for selecting a color of an energy patch coating member, a step for attaching an energy patch at the selected acupuncture point, with the energy patch being selected based on the compass direction energy and the color energy, and a step for fixing an energy patch at a skin of a human body by attaching a certain adhering member on the energy patch.

FIGS. 12A through 12C are views illustrating a method for curing common cold using an energy patch according to the present invention. When a person has a common cold, immunity weakens or the functions of internal organs and lugs may not operate properly. In this case, according to a preferred embodiment of the present invention, the seven energy patches according to a preferred embodiment of the present invention are advantageously attached at corresponding acupuncture points for thereby allowing the acupuncture points to work properly. The method for curing a common cold patient comprises a step for determining a compass direction energy based on 12 zodiac signs by recognizing a patient's age, a step for selecting a color of the energy patch coating member, a step for determining the positions of acupuncture points (Dachu acupuncture point, Pashu acupuncture point, and Pungmun acupuncture point) corresponding to a common cold curing, and a step for attaching an energy patch of the present invention at selected seven acupuncture points.

Here, according to the positions of seven acupuncture points, there are a Dachu acupuncture point as formed at a center of the third cervical vertebral from a back spine of FIG. 12A, a Pungmun acupuncture point at both sides of the sixth cervical vertebral, a Pungmun acupuncture point at a lower side of a middle finger of a left hand of FIG. 12B, and a Pungmun acupuncture point at a lower side of a middle finger of a right hand of FIG. 12C. The energy patches according to the present invention are attached to the above searched acupuncture points. Here, the corner directions of each energy patch and the colors of the color coating members are properly selected for thereby obtaining a significant treatment effect.

FIGS. 13A through 13C are views for describing a method for curing fatigue using an energy patch according to the present invention.

When a person feels fatigue, the person may have a sleeplessness or mental problem due to a stress, anxiety, disease, etc. In this case, five energy patches are preferably attached at five acupuncture points for thereby allowing the corresponding acupuncture points to work properly.

The method for curing a fatigue patient comprises a step for determining a compass direction energy based on 12 zodiac signs by recognizing a patient's age(lunar calendar age), a step for selecting a color of the energy patch coating member, a step for determining the positions of acupuncture points (Sobu acupuncture point, Eojae acupuncture point, Taeyeon acupuncture point, Hanggan acupuncture point, Bokryu acupuncture point) corresponding to a fatigue curing, and a step for attaching an energy patch of the present invention at the selected acupuncture points.

According to the positions of five acupuncture points, there are a Sobu acupuncture point formed at a lower end of a little finger on a left hand palm of FIG. 13A, an Eojae acupuncture point formed at a lower end of a thumb finger, a Taeyeon acupuncture point formed at between a thumb toe and a neighboring toe at a left foot of FIG. 13B, and a Bokryu acupuncture point formed between a thumb toe of a left foot and a neighboring toe of FIG. 13B. The above acupuncture points are located in the above manner, and the energy patches are attached. In this case, it is needed to select the corner directions of the lower direction of each energy patch and the colors of the color coating members for thereby achieving a significant effect accordingly.

FIGS. 14A through 14D are views for describing a method for curing an atopic dermatitis patient using an energy patch according to the present invention. When a person has atopic dermatitis, the energy patch according to the present invention is attached for thereby curing atopic dermatitis for an appetite control therapy. According to a preferred embodiment of the present invention, energy patches are attached at two Jungbu acupuncture points of a chest, five acupuncture points of feet and toes, and four acupuncture points of hands, respectively, for thereby allowing the circulations of corresponding acupuncture points to smoothly activate.

The method for curing atopic dermatitis comprises a step for determining a compass direction energy based on 12 zodiac signs by recognizing a patient's age(lunar calendar age), a step for selecting a color of the energy patch coating member, a step for determining the positions of acupuncture points corresponding to an atopic dermatitis curing, and a step for attaching the energy patches of the present invention at the selected eleven acupuncture points.

The positions of the eleven acupuncture points are two Jungbu acupuncture points formed at both sides of a chest of FIG. 14A, a Taebaek acupuncture point formed at a foot sole of FIG. 14B, a Sanggu acupuncture point formed at an ankle bone, a Gokchun acupuncture point formed at knees, an Eojae acupuncture point formed at a lower side of a center thumb finger of a right hand of FIG. 14C, a Sobu acupuncture point formed at a lower side of a little finger, and an Eunbaek acupuncture point and a Daedon acupuncture point formed at right toes of FIG. 14D. Here, the corner directions of each energy patch and the colors of the color coating members are properly selected for thereby obtaining a significant treatment effect.

FIGS. 15A through 15C are views illustrating a method for curing bronchial asthma using an energy patch according to the present invention.

When a person has bronchial asthma, it is needed to cure by attaching an energy patch according to the present invention. According to a preferred embodiment of the present invention, the energy patches according to the present invention are attached at two Jungbu acupuncture points formed at both sides of a chest, a foot sole, and three acupuncture points formed at a right hand, respectively, for thereby allowing the acupuncture points to activate.

The method for curing a bronchial asthma patient according to the present invention comprises a step for determining a compass direction energy based on 12 zodiac signs by recognizing a patient's age(lunar calendar age), a step for selecting a color of the energy patch coating member, a step for determining the positions of acupuncture points corresponding to a common cold and bronchial asthma curing, and a step for attaching the energy patches of the present invention at the selected six acupuncture points.

The positions of the six acupuncture points are two Jungbu acupuncture points formed at both sides of a chest of FIG. 15A 677, a Taebaek acupuncture point formed at a foot sole of FIG. 15B, an Eojae acupuncture point formed at a lower side of a thumb finger of FIG. 15C, a Sobu acupuncture point formed at a lower end of a little finger, and a Chuntaek acupuncture point formed at an inner side of an elbow. Here, the corner directions of each energy patch and the colors of the color coating members are properly selected for thereby obtaining a significant treatment effect.

FIGS. 16A through 16E are views for describing a method for curing constipation using an energy patch according to the present invention. When a person has constipation, it occurs when the person has a problem at stomach, small intestine or big intestine. In this case, it is possible to cure by attaching an energy patch according to the present invention. According to a preferred embodiment of the present invention, energy patches are attached at two acupuncture points formed at the left and right portions about a center portion of the stomach, two acupuncture points formed at a right hand wrist, an acupuncture point formed at a right arm, two acupuncture points formed at a left wrist, an acupuncture point formed at a little toe of a left foot, and an acupuncture point formed at a right foot ankle, respectively.

The method for curing a constipation patient according to the present invention comprises a step for determining a compass direction energy based on 12 zodiac signs by recognizing a patient's age(lunar calendar age), a step for selecting a color of the energy patch coating member, a step for determining the positions of acupuncture points corresponding to a constipation curing, and a step for attaching the energy patches of the present invention at the selected seven acupuncture points.

The positions of the seven acupuncture points are two Chunchu acupuncture points formed at both sides of a navel of FIG. 16A, a Yanggok acupuncture point and a Yangjae acupuncture point formed at an arm wrist of a right hand of FIG. 16B, a Gokji acupuncture point formed at an elbow, a Yanggok acupuncture point and a Yangjae acupuncture point formed at an arm wrist of a left hand of FIG. 16C, a Samri acupuncture point formed at a knee of a right leg of FIG. 16D, and a Sokgol acupuncture point formed at a little toe of a left foot of FIG. 16E. The corresponding acupuncture points are located in the above manner, and the energy patches are attached. Here, the corner directions of each energy patch and the colors of the color coating members are properly selected for thereby obtaining a significant treatment effect.

As described above, the energy patches according to the present invention may be applied for recognizing disease nature, measurement and control of too much and shortage of energy, and disease prevention. The biological energy may be activated based on a pyramid effect of a certain shape of the energy patch for thereby treating an adult disease and inveterate disease.

Since the compass direction energy and chromatic energy of a human body are considered when the energy patch according to the present invention is attached at a certain portion of the human body, an acupuncture effect can be obtained along with a magnetic therapy effect for thereby enhancing health by controlling energy.

In the present invention, since an energy patch is simply attached on a skin of a user, the user does not feel any refusal and fear while obtaining the same effects as needles as compared to the conventional art in which the user feels fear due to pain when the user receives an acupuncture treatment at an oriental medical hospital.

Since the thickness of the energy patch according to the present invention is less than 0.8 mm, very thin, it is very easy to attach the energy patch on a skin.

Since the energy patch is attached at an acupuncture point and is maintained for 12 hours and then is detached and then attached after 12 hours, easier attachment and detachment are necessary.

Since the energy patch according to the present invention may be attached on a base sheet like the conventional patch, an ordinary person can easily attach or detach the energy patch according to the present invention by locating the acupuncture points. So, a desired economic effect can be easily achieved.

In particular, since the energy patch according to the present invention is used in consideration with the compass direction energy and chromatic energy, which were not considered in the conventional magnetic needle or common needle art, a biological energy can be advantageously activated, so that it is possible to obtain an excellent disease therapy effect.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

What is claimed is:

1. In an energy patch for controlling energy and blood based on a tonification and sadation method with respect to energy and blood at meridians of a human body, an energy patch for controlling energy and blood of a human body, comprising:
a rubber alloy magnet (11) which has a permanent magnet characteristic and is formed in a plane shape with one sharp corner being formed in a lower direction of the same, and at least one sharp corner being formed in an upper direction of the same; and
a color coating member (12) which is color-printed on at least one surface of the surfaces of the rubber alloy magnet (11).

2. The patch of claim 1, further comprising an adhering member (13) for attaching at an acupuncture point of a human body.

3. The patch of claim 1, further comprising a fixing means (20) for attaching at an acupuncture point of a human body.

4. The patch of claim 1, wherein said rubber alloy magnet (11) forms one sharp corner (101) in a lower direction of the same, and a sharp center corner (103) is formed in an upper direction of the same, and two sharp corners (102) and (104) are spaced-apart and formed at both sides of the center corner (103).

5. The patch of claim 1, wherein said rubber alloy magnet (11) forms one sharp corner (111) in a lower direction of the same, and sharp corners (112) and (113) are symmetrically formed at both sides of an upper direction of the same.

6. The patch of claim 1, wherein said rubber alloy magnet (11) forms one sharp corner (121) in a lower direction of the same, and two corners (122) and (123) are spaced-apart and formed at both sides of the center corner, and sharp corners (124) and (125) are symmetrically formed at both sides of an upper direction of the same.

7. The patch of claim 1, wherein said rubber alloy magnet (11) forms one sharp corner (141) in a lower direction of the same, and sharp corners (142) and (143) are symmetrically formed at both sides of an upper direction of the same.

8. The patch of claim 1, wherein a color of said color coating member (12) is selected among white, black, blue and red colors based on a corresponding zodiac sign among twelve zodiac signs of a human body.

9. The patch of claim 8, wherein when selecting the color of the color coating member (12), the tiger, horse and dog zodiac signs are provided with a black color, respectively, and the pig, rabbit and sheep zodiac signs are provided with a white color, respectively, and the monkey, rat and dragon zodiac signs are provided with a red color, respectively, and the snake, chicken and cow zodiac signs are provided with a blue color, respectively.

10. The patch of claim 1, wherein a common permanent magnet, cloth or paper may be used instead the rubber alloy magnet (11).

11. The patch of claim 1, wherein the thickness of said rubber alloy magnet (11) is within a range of 0.5 mm to 5.0 mm.

12. In a method for curing a disease of a person using an energy patch for controlling energy and blood of a human body, a therapy method using an energy patch for controlling energy and blood of a human body, comprising:
a step for determining a compass direction energy based on twelve zodiac signs with respect to a patient's lunar calendar age;
a step for selecting a color of an energy patch coating member;
a step for determining an acupuncture point and a position of the acupuncture point for curing a corresponding disease;
a step for attaching an energy patch at the selected acupuncture point, with the energy patch being selected based on the compass direction energy and the color energy; and
a step for fixing an energy patch at a skin of a human body by attaching a certain adhering member on the energy patch.

13. The method of claim 12, wherein an acupuncture point for curing a common cold patient corresponds to a Dachu acupuncture point, a Pashu acupuncture point and a Pungmun acupuncture point, respectively, and the Dachu acupuncture point is located at a center of a third cervical vertebral from a back neck, and the Pungmun acupuncture point is located at both sides of a sixth cervical vertebral, and the Pashu acupuncture point is formed at both sides of a seventh cervical vertebral, and the Pungmun acupuncture point is located at a lower side of a middle finger of a left hand and a lower side of a middle finger of a right hand.

14. The method of claim 12, wherein when a fatigue patient is cured, the acupuncture points are a Sobu acupuncture point, an Eojae acupuncture point, a Taeyeon acupuncture point, a Hanggan acupuncture point and a Bokryu acupuncture point, and the Sobu acupuncture point is located at a lower end of a little finger of a left hand palm, and the Eojae acupuncture point is located at a lower end of a thumb finger, and the Taeyeon acupuncture point is located at a wrist, and the Hanggan acupuncture point is located between a thumb toe of a left foot and a neighboring toe, and the Bokryu acupuncture point is located at an inner upper side of an ankle.

15. The method of claim 12, wherein when an atopic dermatitis patient is cured, the acupuncture points are a Jungbu acupuncture point, a Taebaek acupuncture point, a Sanggu acupuncture point, a Gokchun acupuncture point, an Eojae acupuncture point, a Sobu acupuncture point, an Eunbaek acupuncture and a Daedon acupuncture point, and the Jungbu acupuncture point is located at both sides of a chest, and the Taebaek acupuncture point is located at a foot sole, and the Sanggu acupuncture point is located at a portion below an ankle bone, and the Gokchun acupuncture point is located at a knee, and the Eojae acupuncture point is located at a lower side of a thumb finger of a right hand, and the Sobu acupuncture point is located at a lower side of a little finger, and the Eunbaek acupuncture point is located at a right side of a thumb toe of a right side, and the Daedon acupuncture point is located at a left side of a right thumb toe.

16. The method of claim 12, wherein when a bronchial asthma patient is cured, the acupuncture points are a Jungbu acupuncture point, a Taebaek acupuncture point, an Eojae acupuncture point, a Sobu acupuncture point, and a Chuktaek acupuncture, and the Jungbu acupuncture point is located at both sides of a chest, and the Taebaek acupuncture point is located at a foot sole, and the Sanggu acupuncture point is located at a portion below an ankle bone, and the Gokchun acupuncture point is located at a knee, and the Eojae acupuncture point is located at a lower side of a thumb finger of a right hand, and the Sobu acupuncture point is located at a lower side of a little finger, and the Chuktaek acupuncture point is located at an inner side of an elbow.

17. The method of claim 12, wherein when a constipation patient is cured, the acupuncture points are a Chunchu acupuncture point, a Yanggok acupuncture point, a Yangjae acupuncture, a Samri acupuncture point, and a Sokgol acupuncture point, and the Chunchu acupuncture point is located at both sides of a navel, and the Yanggok acupuncture point is located at a right side of an arm wrist of a right hand, and the Yangjae acupuncture point is located at a left side of an arm wrist of a right hand, and the Gokji acupuncture point is located at an elbow, and the Yanggok acupuncture point is located at a left side of an arm wrist of a left hand, and the Yangjae acupuncture point is located at a left side of an arm wrist of a right hand, and the Gokji acupuncture point is located at an elbow, and the Yanggok acupuncture point is located at a left side of an arm wrist of a left hand, and the Yangjae acupuncture point is located at a right side of an arm wrist of a left hand, and the Samri acupuncture point is located at a knee of a right leg, and the Sokgok acupuncture point is located at a little toe of a left foot.
